(19) United States
(12) Patent Application Publication
(10) Pub. No.: US 2026/0335930 A1
(43) Pub. Date: Oct. 29, 2026

(54) MAGNETIC BEAD DISTRIBUTION METHOD AND MAGNETIC BEAD DISTRIBUTION DEVICE

(71) Applicant: KAWASAKI INSTITUTE OF INDUSTRIAL PROMOTION, Kawasaki-shi, Kanagawa (JP)

(72) Inventors: Shingo UENO, Kawasaki-shi (JP); Shoichi TSUCHIYA, Kawasaki-shi (JP)

(73) Assignee: KAWASAKI INSTITUTE OF INDUSTRIAL PROMOTION, Kawasaki-shi, Kanagawa (JP)

(21) Appl. No.: 19/106,265

(22) PCT Filed: Aug. 30, 2023
(86) PCT No.: PCT/JP2023/031502

§ 371 Date: Feb. 25, 2025

(30) Foreign Application Priority Data
Aug. 31, 2022 (JP) 2022-138140

(51) Int. Cl. B05D 1/42; B05C 11/04

(52) U.S. Cl. CPC B05D 1/42; B05C 11/045

(57) ABSTRACT

A substrate having a main surface on which a plurality of accommodation parts are formed, a wiping member having a shape that follows that of the main surface, and a magnet disposed on the side of the substrate that is opposite from the wiping member. First, the wiping member is moved forward, whereby a hydrophilic liquid containing a plurality of magnetic beads is spread onto the main surface. The magnet is moved forward together with the wiping member while magnetic force is caused to act on the magnetic beads, whereby the magnetic beads and the hydrophilic liquid are accommodated in the accommodation parts. Second, a hydrophobic liquid arranged on the movement-direction rear side of the wiping member is spread onto the main surface due to the movement of the wiping member, whereby the surface of the hydrophilic liquid within the accommodation parts is covered by the hydrophobic liquid.

### TECHNICAL FIELD

The present invention relates to a magnetic bead distribution method and a magnetic bead distribution device.

Priority is claimed on Japanese Patent Application No. 2022-138140, filed on Aug. 31, 2022, the content of which is incorporated herein by reference.

### BACKGROUND ART

There is a method for immobilizing a biomolecule on a bead for the purpose of observing and detecting the function of a biomolecule. To observe and detect the function of a biomolecule immobilized on a bead, the bead is distributed into an arrayed microwell, for example. Generally, since the sizes of a bead and a microwell for the above purpose is on the micrometer order, it is inefficient to manipulate and place the bead into a microwell one by one. Therefore, a method that distributes multiple beads into multiple microwells at once is employed. A method for observing and detecting the function of a biomolecule involves sealing multiple microwells, into which the bead is distributed, with oil to form closed space. The reaction within these closed space serve as an indicator for observing and detecting the function of the biomolecule on the bead.

A method for forming an oil-sealed bead-containing microwell array include, for example, the following methods (1) and (2):
(1) Flowing an aqueous solution containing a bead into the space between a lower layer and an upper layer in which housing chamber is formed. This introduces the bead-containing aqueous solution into the housing section. Then, introducing oil into the space to seal the housing section with oil (see, for example, Patent Document 1).
(2) Spreading the bead-containing aqueous solution onto the microwell array, layering oil on top of it, and then covering the microwell array with a plate or sheet to seal the microwell.

### CITATION LIST

### Patent Literature

Patent Literature 1: Japanese Patent No. 5337324

### SUMMARY OF INVENTION

### Technical Problem

After observing and detecting the function of a biomolecule in multiple microwells, it may become necessary to perform secondary analysis on the aqueous solution in the microwell or on the biomolecule on the bead.

However, in the aforementioned techniques, the upper surface of the microwell array is covered, which may make it difficult to extract sample (such as beads and aqueous solutions) from the microwell (housing chamber) using capillary or similar tool.

One aspect of the present invention aims to provide a magnetic bead distribution method and a magnetic bead distribution device that are excellent in term of ease of handling sample within the housing chamber.

### Solution to Problem

The present invention includes the following aspects.
[1] A method for distributing a magnetic bead, comprising: a substrate having a main surface on which a plurality of housing chamber is formed therein, a wiping member having a shape that conforms to said main surface, and a magnet placed on a side of said substrate that is opposite from said wiping member; wherein a step of distributing a magnetic bead and a hydrophilic liquid in said housing chamber, by moving said wiping member forward along said main surface to spread a hydrophilic liquid containing plurality of magnetic beads on said main surface, and simultaneously moving said wiping member forward while applying a magnetic force to said magnetic beads, thereby; and a step of covering a surface of said hydrophilic liquid in said housing chamber with a hydrophobic liquid, by spreading a hydrophobic liquid arranged on the rear side of the moving direction of said wiping member onto said main surface by moving said wiping member.
[2] The method for distributing the magnetic bead according to [1], wherein said main surface is hydrophobized, and said inner surface of said housing chamber is hydrophilized.
[3] The method for distributing the magnetic bead according to [1], wherein said wiping member is cylindrical with a central axis along said main surface.
[4] The method for distributing the magnetic bead according to [3], wherein said wiping member comprises a cylindrical main body and a covering layer formed on said outer circumferential surface of said main body and having a hardness lower than that of said outer circumferential surface.
[5] The method for distributing the magnetic bead according to any one of [1] to [4], further comprises a step of forming an annular groove surrounding said housing chamber and placing a liquid stacking frame surrounding said housing chamber on said main surface; a step of forming a hydrophobic liquid layer made of a hydrophobic liquid on said main surface within said liquid stacking frame; and a step of introducing a hydrophilic liquid into said annular groove and causing said hydrophilic liquid to overflow said annular groove to form a hydrophilic liquid layer on said hydrophobic liquid layer.
[6] A magnetic bead distribution device, comprising a distribution magnetic bead into a plurality of housing chamber of a substrate having a main surface on which said housing chamber are formed, wherein a wiping member having a shape that conforms to said main surface, and a magnet placed on a side of said substrate that is opposite from said wiping member, wherein said wiping member is movable forward along said main surface carrying a hydrophilic liquid containing a plurality of magnetic bead, and said magnet is simultaneously movable forward along said wipe member while exerting a magnetic force on said magnetic bead.

### Effects of Invention

According to one aspect of the present invention, it is possible to provide a magnetic bead distribution method and a magnetic bead distribution device that are excellent in terms of ease of handling sample within the housing chamber.

### BRIEF DESCRIPTION OF DRAWINGS

FIG. 1 The front sectional view of a magnetic bead distribution device and an array substrate used in the magnetic bead distribution method according to the embodiment.

FIG. 2 The partial plan view of the array substrate used in the magnetic bead distribution method according to the embodiment.

FIG. 3 The side sectional view of the magnetic bead distribution device and an array substrate used in the magnetic bead distribution method according to the embodiment.

FIG. 4 The explanatory diagram of the magnetic bead distribution method according to the embodiment.

FIG. 5 The explanatory diagram of the magnetic bead distribution method according to the embodiment.

FIG. 6 The sectional view of the liquid stacking frame and array substrate used for forming the hydrophilic liquid layer.

FIG. 7 The explanatory diagram of the method for forming the hydrophilic liquid layer.

FIG. 8 The sectional view of the liquid stacking frame.

FIG. 9 The microscope images of the array substrate in Example 1, in which DNA-immobilized magnetic beads and a fluorescent aqueous solution are distributed and sealed in the microwells. (A) is a bright-field image. (B) is a fluorescence image.

FIG. 10 The illustration showing the procedure for stacking water on the oil layer on the surface of the array substrate using a water-stacking frame in Example 2.

FIG. 11 (A) The image at the start of the test when no water layer is stacked on the oil layer. (B) The image taken 10 minutes after the start of the test.

FIG. 12 (A) The image at the start of the test when a water layer is stacked on the oil layer. (B) The image taken three days after the start of the test.

### DESCRIPTION OF EMBODIMENTS

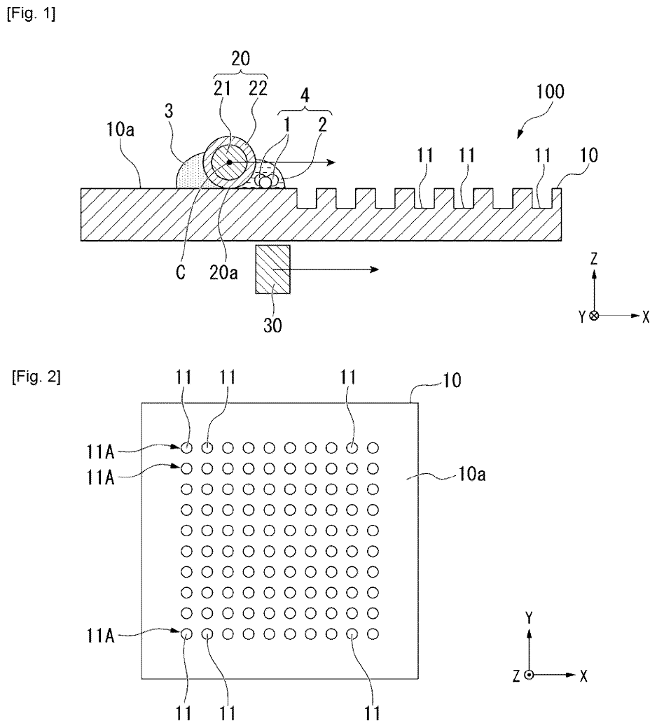

FIG. 1 is a front sectional view of the magnetic bead distribution device 100 and the array substrate 10 used in the magnetic bead distribution method according to the embodiment.

FIG. 2 is a partial plan view of the array substrate 10.

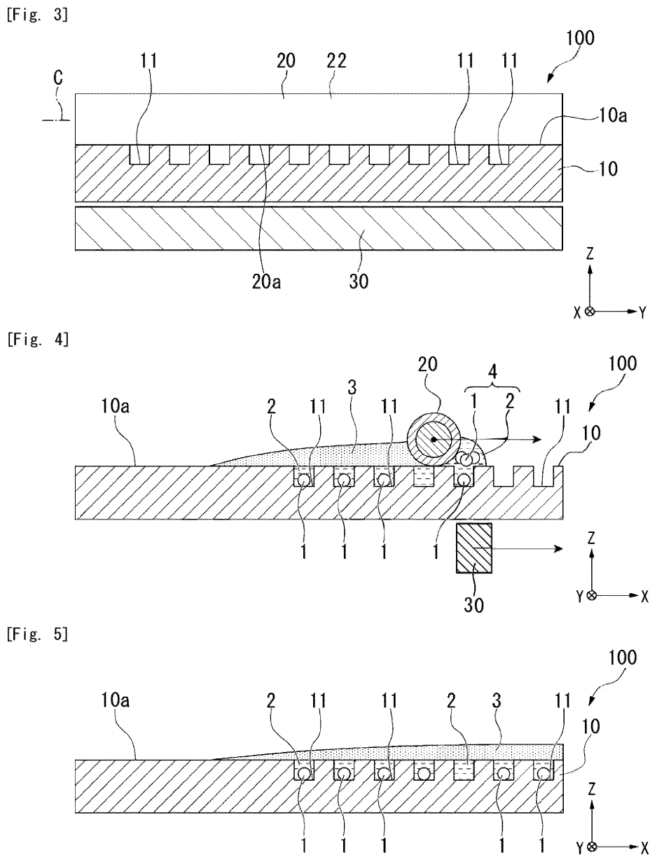

FIG. 3 is a side sectional view of the magnetic bead distribution device 100 and the array substrate 10.

As shown in FIG. 1, the magnetic bead distribution device 100 comprises a wiping member 20 and a magnet 30. The magnetic bead distribution device 100 distributes magnetic beads onto the array substrate 10.

### Substrate

The array substrate 10 has a plurality of housing chamber (microwell) 11 formed on its main surface 10a, which is one of its surface. The array substrate 10 is, for example, rectangular in plan view (see FIG. 2). The array substrate 10 is plate-shaped, and the main surface 10a is flat. The array substrate 10 is an example of a “substrate.”

It is preferable for the array substrate 10 to be transparent. If the array substrate 10 is transparent, it becomes easier to observe the sample within the housing chamber 11. Note that the light transmittance of the array substrate 10 is not particularly limited.

As shown in FIG. 2, an X direction is the direction along a pair of opposing sides of the array substrate 10. One direction in the X direction (right direction in FIGS. 1 and 2) is the +X direction. The opposite direction to the +X direction (left direction in FIGS. 1 and 2) is the −X direction. The Y direction is in the plane along the main surface 10a and is orthogonal to the X direction. The Z direction is orthogonal to both the X and Y directions. A plan view is viewed from the Z direction. Hereafter, the vertical positional relationship is tentatively defined in accordance with FIG. 1. The array substrate 10 is positioned with its main surface 10a facing upwards. The main surface 10a is horizontal.

The housing chamber 11 is recesses formed on the main surface 10a. The housing chamber 11 is capable of containing sample. Since the array substrate 10 has a plurality of housing chamber 11, it has the advantage of being able to process multiple samples simultaneously.

An internal dimension (e.g., inner diameter) and a depth of the housing chamber 11 are determined according to the size of the magnetic bead 1 to be contained. It is desirable for the internal dimensions and depth of the housing chamber 11 to be slightly larger than the average particle size of the magnetic bead 1. For example, the internal dimension and depth of the housing chamber 11 may be greater than one time but less than two times the average particle size of the magnetic bead 1. This makes it easier for each housing section 11 to accommodate only one magnetic bead 1.

The shape of the housing chamber 11 in plan view is, for example, circular. The housing chamber 11, for example, has a cylindrical internal space.

It should be noted that the shape of the housing chamber 11 is not particularly limited. The shape of the housing chamber 11 in plan view may be polygonal, such as rectangular or hexagonal. The housing chamber 11 may also have a shape in which the inner diameter decreases in the depth direction

The plurality of housing chamber 11 is arranged in a two-dimensional matrix, for example, in multiple rows and multiple columns. The plurality of housing chamber 11 may form multiple groups of housing chamber 11A. Each group of housing chamber 11A is composed of multiple housing chamber 11 arranged at regular intervals along the X direction. The multiple groups of housing chamber 11A are arranged at regular intervals along the Y direction. In FIG. 2, the housing chamber 11 in adjacent groups of housing chamber 11A in the Y direction is positioned in alignment along the Y direction, but the housing chamber 11 in adjacent groups of housing chamber 11A does not necessarily have to be aligned in the Y direction. For example, the housing chamber 11 may be arranged in a staggered pattern in plan view.

It should be noted that the number and arrangement of the plurality of housing chamber is not particularly limited. For example, the number of groups of housing chamber may be one. That is, the substrate may have all the housing chamber arranged in a single row. Two or more of the plurality of housing chambers are formed at different positions in the X direction.

The material for the array substrate 10 includes resin, glass, quartz, and the like. Example of resin includes cycloolefin polymer (COP), polystyrene (PS), polycarbonate (PC), cyclic olefin copolymer (COC), polydimethylsiloxane (PDMS), polyethylene terephthalate (PET), acrylic resin (such as PMMA), and the like. The array substrate 10 may be a laminated structure comprising a flat base material and a well structure formed on the base material. The well (housing chamber) is formed in the well structure. The base material is made of glass, quartz, silicon wafer, and the like. The well structure is formed of photoresist, and the like.

It is desirable for the inner surface of the housing chamber 11 to be hydrophilized. Hydrophilization treatment method includes oxygen plasma irradiation and ultraviolet-ozone treatment. When the inner surface of the housing chamber 11 is hydrophilized, it is easy to introduce the hydrophilic liquid 2 into the housing chamber 11.

It is desirable for the main surface 10a to be hydrophobized. Treatment agent for hydrophobizing the main surface 10a of the array substrate 10 includes hexamethyldisilazane, organochlorosilane, polyorganosiloxane, and the like. For example, an array substrate 10 having a hydrophobized main surface 10a can be obtained by pressing a polypropylene film vapor-deposited with hexamethyldisilazane (HMDS) onto the main surface 10a to transfer the HMDS to the main surface 10a.

### Magnetic Bead Distribution Device

As shown in FIG. 1, the wiping member 20 is cylindrical, having a central axis C along the Y direction. The side surface (outer peripheral surface) of the wiping member 20 is in contact with the main surface 10a of the array substrate 10. Because the wiping member 20 is cylindrical, it has a large contact area with the main surface 10a. Therefore, the liquid stopping ability of the wiping member 20 can be enhanced.

As shown in FIG. 3, the lowermost part 20a of the wiping member 20 is linear along the Y direction when viewed from the X direction. Because the lowermost part 20a is linear, the wiping member 20 can contact the flat main surface 10a without any gaps. Therefore, it can be said that the wiping member 20 has a shape conforming to the main surface 10a of the array substrate 10. The lowermost part 20a of the wiping member 20 covers the openings of the housing chamber 11 in the side sectional view (a sectional view along the Y and Z directions) of the array substrate 10.

As shown in FIG. 1, the wiping member 20 includes a main body 21 and a covering layer 22. The main body 21 is cylindrical with a central axis C. The main body 21 is composed of, for example, metal, resin, or similar materials.

The covering layer 22 is formed on the outer peripheral surface of the main body 21. The covering layer 22 covers the entire outer peripheral surface of the main body 21. The covering layer 22 has a lower hardness than the outer peripheral surface of the main body 21. Example of hardness includes durometer hardness (in accordance with JIS K6253 or JIS K7215). It is preferable for the covering layer 22 to be an elastic body. The covering layer 22 is formed of, for example, rubber (such as silicone rubber), thermoplastic resin, thermoplastic elastomer, thermosetting elastomer, and the like. The physical property of the covering layer 22 is selected in consideration of adhesion to the main surface 10a and liquid-stopping ability. The covering layer 22 can be formed, for example, by covering the main body 21 with a silicone rubber tube (silicone tube).

The magnet 30 is composed of, for example, rare-earth magnet material (e.g., neodymium iron boron (NdFeB), samarium cobalt (SmCo)), ceramic magnet material (e.g., strontium ferrite), and other magnetic material (e.g., iron, cobalt, nickel, their alloys, and oxides). Neodymium magnet (NdFeB) is preferred for the magnet 30.

The shape of the magnet 30 is determined so that it can exert magnetic force on the magnetic bead 1 contained in the bead suspension 4 on the main surface 10a. As shown in FIG. 3, the magnet 30 can be, for example, prismatic, extending in the Y direction. Since the magnet 30 has a length that extends over the entire length of the array substrate 10 in the Y direction, it can exert magnetic force on the magnetic beads 1 over a wide range in the Y direction.

The magnet 30 may have one of its ends (either the upper end or the lower end) as the N pole and the other end as the S pole. The magnet 30 is not limited to being prismatic; it can also be cylindrical, for example. The magnet 30 can also be an electromagnet.

The magnet 30 is positioned on the opposite side of the array substrate 10 from the wiping member 20. Specifically, the magnet 30 is located on the lower surface side of the array substrate 10. The magnet 30 is at a height position close to the lower surface of the array substrate 10. The magnet 30 can move in the +X direction (rightward in FIG. 1).

### Magnetic Bead Distribution Method

### First Embodiment

Next, the magnetic bead distribution method according to the first embodiment will be described. The magnetic bead distribution method of this embodiment performs the first step (housing the magnetic beads into the housing chamber) and the second step (covering the hydrophilic liquid within the housing chamber with a hydrophobic liquid) in a single operation.

### First Step: Housing Magnetic Beads Into Housing Chamber

As shown in FIG. 1, the wiping member 20 is placed on the main surface 10a of the array substrate 10. The wiping member 20 contacts the main surface 10a without any gaps along its length direction (Y direction). The direction of movement of the wiping member 20 is the +X direction (rightward in FIG. 1). The +X direction is the “forward” (first direction). The −X direction is the “backward” direction.

On the main surface 10a, in front of the wiping member 20, a hydrophilic liquid 2 containing a plurality of magnetic bead 1 is placed.

The magnetic bead 1 is, for example, immobilized with a biomolecule such as a nucleic acid, a peptide, or a protein. The magnetic bead 1 is dispersed in the hydrophilic liquid 2. The hydrophilic liquid 2 is, for example, water or an aqueous solution. The hydrophilic liquid 2 containing the magnetic bead 1 is referred to as the “bead suspension 4.” The bead suspension 4 is in contact with the front surface of the wiping member 20. The magnetic bead 1 is particle containing a magnetic material. Examples of magnetic material includes iron, nickel, cobalt, or their oxides. The average particle size of the magnetic bead 1 may be, for example, 1 μm to 10 μm. The preferred average particle size of the magnetic bead 1 is 1 μm to 5 μm, and more preferably 2 μm to 4 μm.

On the main surface 10a, behind the wiping member 20, a hydrophobic liquid 3 is placed. The hydrophobic liquid 3 is in contact with the rear surface of the wiping member 20.

Example of hydrophobic liquids 3 includes fluorine oil and silicone oil. As for fluorine oil, there is perfluoropolyether (PFPE), perfluoroalkyl ether (PFAE), and perfluoropolyalkyl ether (PFPAE). Other example of hydrophobic liquids 3 includes hydrofluoroether (HFE), mineral oil, saturated hydrocarbon, unsaturated hydrocarbon, aromatic hydrocarbon, and perfluorocarbon.

FIGS. 4 and 5 are explanatory diagrams of the magnetic bead distribution method according to the embodiment.

As shown in FIGS. 4 and 5, the wiping member 20 is moved forward (in the +X direction) along the main surface 10a while maintaining its posture. The wiping member 20 moves forward while maintaining contact with the main surface 10a along its length direction. The forward movement of the wiping member 20 pushes the bead suspension 4 forward and spreads it over the forward area of the main surface 10a. The direction of movement of the wiping member 20 is forward (in the +X direction).

When the wiping member 20 moves forward, the magnet 30 is moved forward (in the +X direction) together with the wiping member 20. The magnet 30 moves forward at the same speed as the wiping member 20. Therefore, the magnet 30 moves forward without changing its relative position to the wiping member 20.

It is desirable for the magnet 30 to be positioned forward of the wiping member 20 in plan view. The magnet 30 exerts a downward magnetic force (attractive force) on the magnetic bead 1 contained in the bead suspension 4 in front of the wiping member 20. The magnet 30 moves forward while exerting magnetic force on the magnetic beads 1. As a result, the magnetic bead 1 is positioned slightly ahead of the wiping member 20 due to the magnet 30, making it easier for them to be introduced into the housing chamber 11 without being interfered with by the wiping member 20.

A portion of the bead suspension 4 is introduced into the housing chamber 11. Since the main surface 10a is hydrophobized and the inner surfaces of the housing chamber 11 is hydrophilized, the bead suspension 4 can easily enter the housing chamber 11. The magnetic beads 1 are, for example, distributed into the housing chamber 11 one by one.

### Second Step: Covering the Hydrophilic Liquid in the Housing Chamber With Hydrophobic Liquid

As shown in FIGS. 1 and 4, the hydrophobic liquid 3 behind the wiping member 20 spreads across the main surface 10a as it follows the forward movement of the wiping member 20. Consequently, the hydrophobic liquid 3 forms a layer that covers the surface of the hydrophilic liquid 2 within the housing chamber 11. This layer of hydrophobic liquid 3 isolates the multiple housing chamber 11 from each other. The bead suspension 4 is sealed within the housing chamber 11.

### Handling of Sample

Operations on the magnetic bead 1 within the housing chamber 11 can be performed from above the housing chamber 11. For example, the operation of an extracting sample from the housing chamber 11 can be performed using an experimental tool such as a capillary, which is brought close to the housing chamber 11 from above. Sample that can be extracted from the housing chamber 11 using tool like a capillary include, for example, magnetic bead 1, hydrophilic liquid 2, bead suspension 4, and the like.

### Effects of the Magnetic Bead Distribution Method and Device of the First Embodiment

The magnetic bead distribution method of this embodiment involves moving the wiping member 20 forward to spread the bead suspension 4 on the main surface 10a while applying magnetic force to the magnetic bead 1 with the magnet 30, moving it forward to house the bead suspension 4 into the housing chamber 11 (First Step). The hydrophobic liquid 3 behind the wiping member 20 is spread across the main surface 10a by the movement of the wiping member 20 and covers the surface of the hydrophilic liquid 2 within the housing chamber 11 (Second Step). The hydrophobic liquid 3 isolates the multiple housing chambers 11 from each other. The bead suspension 4 is sealed within the housing chamber 11.

In the magnetic bead distribution method according to this embodiment, the magnetic bead 1 can be distributed into the housing chamber 11 and the housing chamber 11 can be sealed with the hydrophobic liquid 3 with a single operation of moving the wiping member 20 and the magnet 30 forward. Therefore, the task of distributing the magnetic bead 1 becomes easier. Additionally, since the hydrophilic liquid 2 within the housing chamber 11 is immediately covered with the hydrophobic liquid 3, the exposure time of the hydrophilic liquid 2 in the housing chamber 11 can be minimized. This suppresses the evaporation of the hydrophilic liquid 2.

In the magnetic bead distribution method according to this embodiment, the liquid containing magnetic bead 1 is distributed into the housing chamber 11 while keeping the upper side of the array substrate 10 open. Therefore, unlike the method using the upper layer provided on the array substrate 10 (refer to Japanese Patent No. 5337324), the operation of an extracting sample from the housing chamber 11 can be performed by bringing an experimental tool such as a capillary close to the housing chamber 11 from above. Thus, the ease of a handling sample within the housing chamber 11 can be improved.

The array substrate 10 has a hydrophobized main surface 10a, and the inner surfaces of the housing chamber 11 is hydrophilized, making it easier for the bead suspension 4 to enter the housing chamber 11.

Since the wiping member 20 is cylindrical, it contacts the main surface 10a with a large contact area. This increases the liquid-stopping ability of the wiping member 20. Therefore, it can prevent the bead suspension 4 from leaking to the rear side of the wiping member 20 and the hydrophobic liquid 3 from entering the front side of the wiping member 20.

Since the wiping member 20 has a low-hardness covering layer 22, it can enhance the adhesion of the wiping member 20 to the main surface 10a and improve the liquid-stopping ability of the wiping member 20.

In the magnetic bead distribution device 100 according to this embodiment, the magnetic bead 1 can be distributed into the housing chamber 11 and the housing chamber 11 can be sealed with the hydrophobic liquid 3 with a single operation of moving the wiping member 20 and the magnet 30 forward. Therefore, the task of distributing the magnetic bead 1 becomes easier. Additionally, since the hydrophilic liquid 2 within the housing chamber 11 is immediately covered with the hydrophobic liquid 3, the exposure time of the hydrophilic liquid 2 in the housing chamber 11 can be minimized. This suppresses the evaporation of the hydrophilic liquid 2.

### Magnetic Bead Distribution Method

### Second Embodiment

The magnetic bead distribution method according to this embodiment can perform layer formation of the hydrophobic liquid and the hydrophilic liquid following the previously described magnetic bead distribution method. Specifically, the magnetic bead distribution method according to this embodiment, following the previously described first step (housing the magnetic beads into the housing chamber) and second step (covering the hydrophilic liquid within the housing chamber with a hydrophobic liquid), then performs the third step, fourth step, and fifth step described below.

In the third step, a liquid stacking frame that surrounds the housing chamber is installed on the main surface of the substrate, which has multiple housing chamber formed on its main surface. The liquid stacking frame forms an annular groove around the housing chamber.

In the fourth step, a hydrophobic liquid layer made of hydrophobic liquid is formed on the main surface within the liquid stacking frame.

In the fifth step, a hydrophilic liquid is introduced into the annular groove, and the hydrophilic liquid is allowed to overflow from the annular groove, forming a hydrophilic liquid layer on top of the hydrophobic liquid layer.

The following are detailed descriptions of each step.

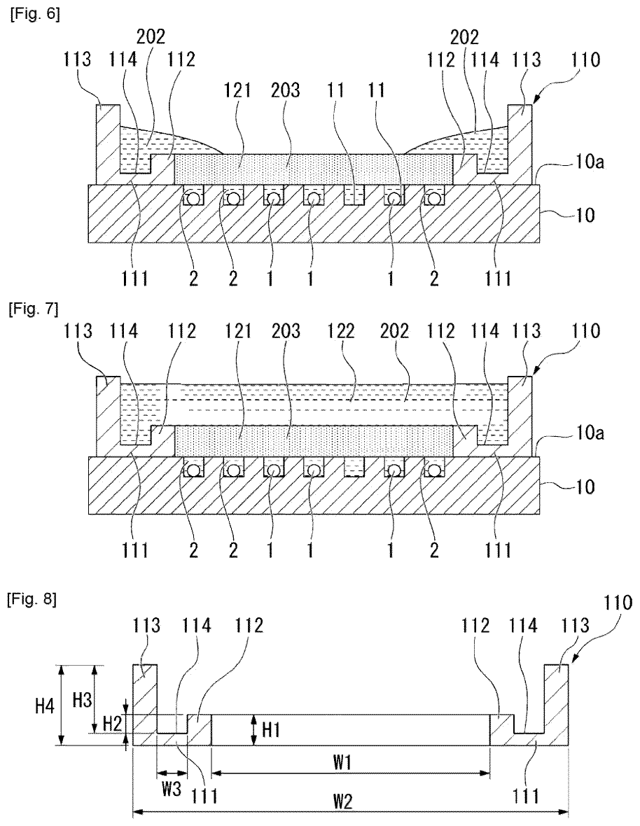

FIG. 6 is a cross-sectional view of the liquid stacking frame 110 and the array substrate 10 used in the magnetic bead distribution method according to this embodiment. FIG. 7 is an explanatory diagram of the liquid layer formation method. FIG. 8 is a cross-sectional view of the liquid stacking frame 110. The region on the main surface 10a of the array substrate 10 where multiple housing chambers 11 are formed is referred to as the “array region.” The array region is the area that collectively includes multiple housing chamber 11. Common configurations with other embodiments are described with the same reference numerals and their explanations are omitted.

### Third Step: Installation of Liquid Stacking Frame

As shown in FIG. 6, the liquid stacking frame 110 is installed on the main surface 10a of the array substrate 10. The housing chamber 11 of the array substrate 10 contains the magnetic bead 1 and the hydrophilic liquid 2, and the housing chamber 11 are sealed with the hydrophobic liquid 3, as per the previously described magnetic bead distribution method.

The liquid stacking frame 110 includes a bottom wall 111, an inner peripheral wall 112, and an outer peripheral wall 113. The bottom wall 111 is formed in an annular shape. The bottom wall 111 can be, for example, circular or rectangular annular. The internal dimension of the bottom wall 111 is determined such that the bottom wall 111 can surround the array region in plan view. The opening of the bottom wall 111 encompasses the array region in plan view. If the bottom wall 111 is circular, its internal dimension is its inner diameter. If the bottom wall 111 is rectangular annular, its internal dimensions are the lengths of the sides of the rectangular opening. The bottom wall 111 is installed on the main surface 10a of the array substrate 10.

The inner peripheral wall 112 is erected on the inner peripheral edge of the bottom wall 111. The inner peripheral wall 112 is cylindrical in shape, corresponding to the shape of the inner peripheral edge of the bottom wall 111. For example, if the bottom wall 111 is annular, the inner peripheral wall 112 becomes cylindrical. The height of the inner peripheral wall 112 from the upper surface of the bottom wall 111 is determined according to the thickness of the hydrophobic liquid layer 121 formed on the main surface 10a. The height of the inner peripheral wall 112 is uniform throughout its entire perimeter.

The outer peripheral wall 113 is erected on the outer peripheral edge of the bottom wall 111. The outer peripheral wall 113 is cylindrical in shape, corresponding to the shape of the outer peripheral edge of the bottom wall 111. For example, if the bottom wall 111 is annular, the outer peripheral wall 113 becomes cylindrical. The inner diameter of the outer peripheral wall 113 is larger than the outer diameter of the inner peripheral wall 112. The outer peripheral wall 113 is located outward from the inner peripheral wall 112. Therefore, an annular groove 114 is formed between the inner peripheral wall 112 and the outer peripheral wall 113.

The height of the outer peripheral wall 113 from the upper surface of the bottom wall 111 (the bottom surface of the annular groove 114) is greater than the height of the inner peripheral wall 112. The height of the outer peripheral wall 113 is determined according to the thickness of the hydrophilic liquid layer 122 formed on the hydrophobic liquid layer 121.

The specific configuration of the liquid stacking frame 110 is shown in FIG. 8. As shown in FIG. 8, the height H1 of the inner peripheral wall 112 from the bottom surface of the bottom wall 111 is, for example, 2 mm. The height H2 of the inner peripheral wall 112 from the bottom surface of the annular groove 114 is, for example, 1.5 mm. The height H3 of the outer peripheral wall 113 from the bottom surface of the annular groove 114 is, for example, 9.5 mm. The height H4 of the outer peripheral wall 113 from the bottom surface of the bottom wall 111 is, for example, 10 mm. The bottom wall 111 surrounds the array region with an annular peripheral area of 1.5 mm in width. Therefore, the internal dimensions of the bottom wall 111, represented by the width W1, are the dimensions of the array region plus 3 mm. The external dimensions of the bottom wall 111, represented by the width W2, are equivalent to the external dimensions of the array substrate 10. The width W3 of the annular groove 114 is the distance between the inner peripheral wall 112 and the outer peripheral wall 113. The width W3 can be determined according to the widths W1 and W2.

Note that while the liquid stacking frame 110 includes the bottom wall 111, the liquid stacking frame may also have a configuration without a bottom wall. In this case, it is preferable that the inner peripheral wall and the outer peripheral wall are connected to each other by one or more connecting portions formed in one or more locations.

### Fourth Step: Formation of Hydrophobic Liquid Layer

By introducing a hydrophobic liquid 203 into the space defined by the main surface 10a and the inner peripheral wall 112, a hydrophobic liquid layer 121 is formed. The hydrophobic liquid layer 121 is formed on the main surface 10a and encompasses all the housing chamber 11. The thickness of the hydrophobic liquid layer 121 is, for example, equal to or smaller than the height of the inner peripheral wall 112. Examples of hydrophobic liquid 203 include fluorine oil and silicone oil. As for fluorine oils, there are perfluoropolyethers (PFPE), perfluoroalkyl ethers (PFAE), and perfluoropolyalkyl ethers (PFPAE). Other examples of hydrophobic liquid 203 include hydrofluoroether (HFE), mineral oil, saturated hydrocarbons, unsaturated hydrocarbons, aromatic hydrocarbons, and perfluorocarbons. The hydrophobic liquid 203 may have a higher viscosity compared to the hydrophobic liquid 3.

### Fifth Step: Formation of Hydrophilic Liquid Layer

As shown in FIGS. 6 and 7, the hydrophilic liquid 202 is introduced into the annular groove 114. Examples of hydrophilic liquid 202 include water or aqueous solution. After the hydrophilic liquid 202 fills the annular groove 114, additional hydrophilic liquid 202 is introduced, causing it to overflow from the annular groove 114 and flow inward over the inner peripheral wall 112. The inflow of the hydrophilic liquid 202 occurs over a wide range in the circumferential direction of the inner peripheral wall 112. For example, the hydrophilic liquid 202 flows inward over the inner peripheral wall 112 around its entire circumference simultaneously.

By further introducing the hydrophilic liquid 202, a hydrophilic liquid layer 122 is formed on top of the hydrophobic liquid layer 121. The hydrophilic liquid layer 122 is formed inside the outer peripheral wall 113. The hydrophilic liquid layer 122 can suppress the evaporation of the hydrophilic liquid 2 in the housing chamber 11 through the hydrophobic liquid layer 121.

### Effects of the Magnetic Bead Distribution Method of the Second Embodiment

According to the magnetic bead distribution method of this embodiment, the inflow of the hydrophilic liquid 202 inside the inner peripheral wall 112 occurs over a wide circumferential range of the inner peripheral wall 112. Therefore, the load applied by the hydrophilic liquid 202 to the hydrophobic liquid layer 121 during inflow is dispersed. This prevents the hydrophilic liquid 202 from locally penetrating the hydrophobic liquid layer 121 and reaching the housing chamber 11. As a result, a hydrophilic liquid layer 122 that covers the entire hydrophobic liquid layer 121 can be formed. This suppresses the evaporation of the hydrophilic liquid 2 in the housing chamber 11. Therefore, a lid member to prevent the evaporation of the hydrophilic liquid 2 is not necessary.

### EXAMPLES

The present invention will be described based on examples. However, the embodiments of the present invention are not limited to the descriptions of these examples.

### Example 1

### <Synthesis of Biotin-Modified DNA>

A PCR reaction solution with the following composition was prepared and PCR was performed for 30 cycles (98 °C for 10 seconds; 55 °C for 5 seconds; 72 °C for 2 minutes). The PCR product was purified using a QIAquick® PCR Purification Column (QIAGEN) to obtain biotin-modified DNA.

### (PCR Reaction Solution)

20 pg/μL template DNA
0.3 μM biotin-modified DNA primer
0.3 μM DNA primer
0.2 μM each dNTP Mix
0.025 U/μL PrimeSTAR® HS polymerase
1× PrimeSTAR® buffer (Takara Bio)

### <Preparation of DNA-Immobilized Magnetic Bead>

Removed 120 μL of the supernatant from streptavidin-modified magnetic beads (MS300/streptavidin, JSR; hereinafter referred to as “magnetic beads”) and washed the magnetic beads with 100 μL of binding buffer (10 mM Tris-HCl, 1 mM EDTA, 1 M NaCl, 0.05% (w/v) Tween® 20, pH 7.4). Suspended the magnetic beads in 170 μL of binding buffer in which 7 pmol of biotin-modified DNA was dissolved, and agitated it at room temperature for 30 minutes. This caused the biotin-modified DNA to bind to the magnetic beads. Then washed the magnetic beads five times with 100 μL of binding buffer. Finally, suspended the magnetic beads in 30 μL of binding buffer.

The concentration of DNA-immobilized magnetic beads in the above suspension was measured using an automatic cell counter (product name: Countess II, Thermo Fisher Scientific). The supernatant from the DNA-immobilized magnetic beads suspension (equivalent to 5×10⁷ DNA-immobilized magnetic beads) was removed, and the DNA-immobilized magnetic beads were suspended in 60 μL of fluorescent aqueous solution (10 μM sodium fluorescein, 50 mM Tris-HCl, pH 7.5). The DNA-immobilized magnetic beads suspension prepared in this way was used for the subsequent section, “Distribution of Beads and Aqueous Solution onto Array Substrate”.

### <Preparation of the Array Substrate>

Nickel-phosphorus was plated to a thickness of approximately 100 μm on the surface of an STAVAX® substrate (30 mm×30 mm×5 mm). Using a cutting tool with a blade width of 6 μm, grid-like grooves were processed into the nickel-phosphorus layer, creating a mold with a cubic structure (4 μm×4 μm×4 μm) arranged in a grid of 1×10⁶ units on a plane (1.2 mm×1.2 mm). This mold was mounted on an injection molding machine, and cycloolefin polymer (trade name: ZEONEX® 480, manufactured by Zeon Corporation) was injected. This process created an array substrate with microwells (4 μm×4 μm×4 μm) arranged in 1×10⁶ units on an area (1.2 mm×1.2 mm) of the substrate surface (30 mm×30 mm×1 mm). Hereinafter, the area of the array substrate where the microwells are arranged is referred to as the “array region.” The area of the array substrate where there are no microwells is referred to as “outside the array region.”

### <Surface Treatment of Array Substrate>

By irradiating the main surface of the array substrate with oxygen plasma, the main surface of the array substrate and the inner surfaces of the microwells were made hydrophilic. Subsequently, a polypropylene film deposited with hexamethyldisilazane (HMDS) was pressed onto the surface of the array substrate, transferring the HMDS onto the substrate surface. The substrate was then treated at 110 °C for 10 minutes to render the substrate surface hydrophobic. Through these operations, an array substrate was produced with hydrophilic inner surfaces of the microwells and a hydrophobic main surface outside the microwells.

### <Distribution of Beads and Aqueous Solution Onto Array Substrate>

A stainless steel rod with a diameter of 3 mm was inserted into a silicone tube (outer diameter 4 mm, inner diameter 2 mm, length 50 mm) to create a wiping member. The wiping member was placed in contact with the area outside the array region of the array substrate. A neodymium magnet (20×5×10 mm) was positioned under the array substrate between the contact part of the wiping member and the edge of the array region.

60 μL of the DNA-immobilized magnetic beads suspension was dropped onto the main surface of the array substrate (in the position ahead of the wiping member: the area between the contact part of the wiping member and the edge of the array region). Additionally, 60 μL of fluorine oil (trade name: Krytox® GPL 104, manufactured by Chemours) was dropped onto the main surface of the array substrate (in the position behind the wiping member).

The wiping member and neodymium magnet were moved forward simultaneously at a speed of 0.1 mm/s and stopped once they passed the array region. This operation distributed the DNA-immobilized magnetic beads and the fluorescent aqueous solution into the microwells and sealed them with fluorine oil.

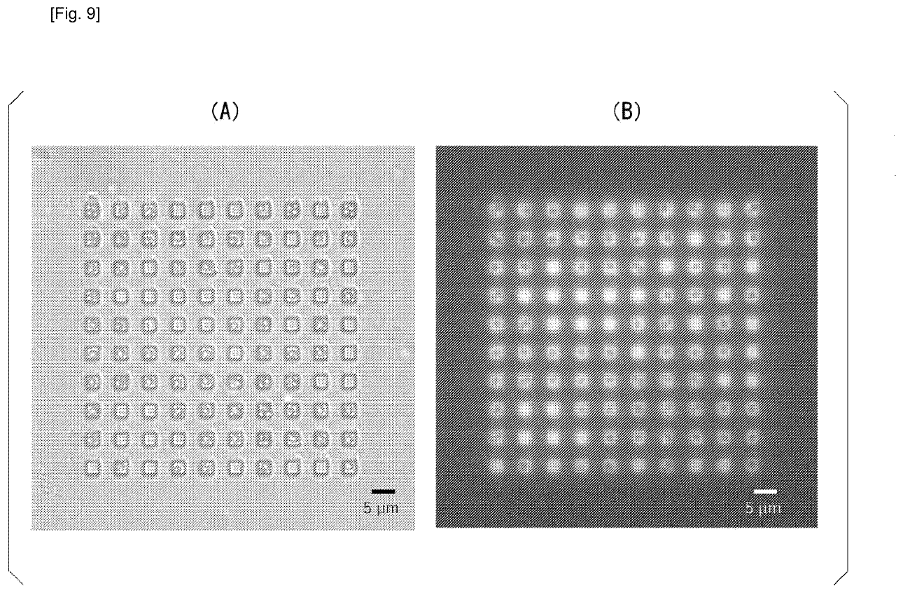

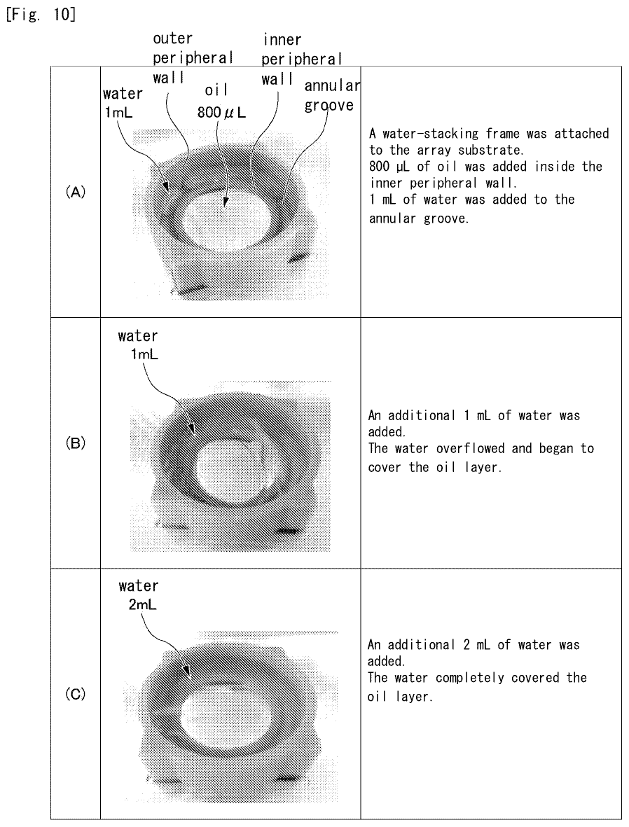

FIGS. 9(A) and 9(B) show microscope images of the array substrate, in which the DNA-immobilized magnetic beads and the fluorescent aqueous solution are sealed by fluorinated oil (FIG. 9(A): bright-field image, FIG. 9(B): fluorescent image). As shown in FIGS. 9(A) and 9(B), the fluorescent aqueous solution was distributed in all the microwells within the array region, and DNA-immobilized magnetic beads were distributed in 67% of the microwells.

### Example 2

### <Formation of Water Layer on Oil Layer of Array Substrate Surface>

As shown in FIG. 10(A), a water-stacking frame (liquid stacking frame) was created using a 3D printer (product name: From2, manufactured by Formlabs). The water-stacking frame was attached to the array substrate containing DNA-immobilized magnetic beads and fluorescent aqueous solution in the microwells. 800 μL of fluorine oil (trade name: Krytox® GPL 107, manufactured by Chemours) was added inside the inner peripheral wall of the water-stacking frame. This formed a hydrophobic liquid layer made of fluorine oil on the main surface of the array substrate.

As shown in FIGS. 10(B) and 10(C), after filling the annular groove between the inner and outer peripheral walls of the water-stacking frame with 1 mL of water, an additional 3 mL of water was added to the annular groove. This caused the water in the annular groove to overflow, forming a layer of water (hydrophilic liquid layer) on top of the fluorine oil (hydrophobic liquid layer) inside the inner peripheral wall.

The retention time of the fluorescent aqueous solution in the microwells was confirmed using a fluorescence microscope.

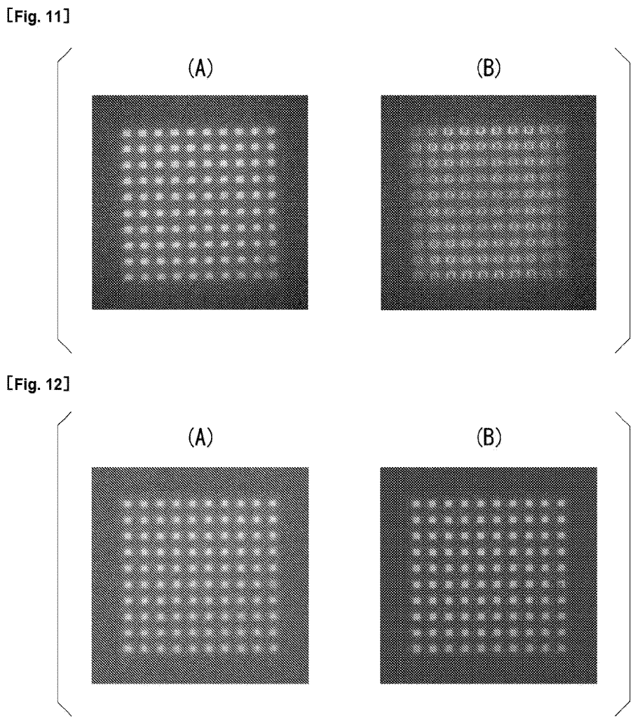

FIG. 11(A) shows the image at the start of the test without stacking a water layer on the oil layer. FIG. 11(B) shows the image 10 minutes after the start of the test. FIG. 12(A) shows the image at the start of the test with a water layer stacked on the oil layer. FIG. 12(B) shows the image 3 days after the start of the test.

As shown in FIGS. 11(A) and 11(B), when a water layer was not stacked on the oil layer, the fluorescent aqueous solution in the microwells disappeared within 10 minutes after the start of the test. As shown in FIGS. 12(A) and 12(B), when a water layer was formed on the oil layer, the fluorescent aqueous solution was retained in the microwells even after 3 days from the start of the test.

The embodiment of the present invention has been described above; however, the configuration and combination thereof in the embodiment is merely example, and addition, omission, substitution, and other modification of the configuration are possible within the scope of the gist of the present invention.

For example, the number of magnetic bead 1 housed in the housing chamber 11 is not limited to one. The number of magnetic bead 1 housed in the housing chamber 11 may be multiple (any number of 2 or more). The number of housed magnetic bead 1 can be adjusted by at least one of the internal dimension (inner diameter) and depth of the housing chamber 11. When the number of magnetic bead 1 housed in the housing chamber 11 is multiple, the number of analyte bead per unit area of the array substrate 10 can be increased. Therefore, screening can be performed efficiently.

The wiping member shown in FIG. 1 is cylindrical, but the shape of the wiping member is not particularly limited. For example, the wiping member may be blade-shaped. While it is preferable for the wiping member to be in contact with the main surface of the substrate, it may also be slightly distanced from the main surface.

The contact angle of water on the hydrophobic surface is, for example, 60 degrees or more. The contact angle of water on the hydrophilic surface is, for example, 40 degrees or less.

### REFERENCE SIGNS LIST

1: Magnetic bead
2, 202: Hydrophilic liquid
3, 203: Hydrophobic liquid
10: Array substrate (substrate)
10a: Main surface
11: Housing chamber
20: Wiping member
21: Main body
22: Covering layer
30: Magnet
100: Magnetic bead distribution device
110: Liquid stacking frame
114: Annular groove
121: Hydrophobic liquid layer
122: Hydrophilic liquid layer
C: Central axis

1. A method for distributing a magnetic bead, comprising:
a substrate having a main surface on which a plurality of housing chamber is formed therein,
a wiping member having a shape that conforms to said main surface, and
a magnet placed on a side of said substrate that is opposite from said wiping member; wherein
a step of distributing a magnetic bead and a hydrophilic liquid in said housing chamber, by
moving said wiping member forward along said main surface to spread a hydrophilic liquid containing plurality of magnetic beads on said main surface, and simultaneously moving said wiping member forward while applying a magnetic force to said magnetic beads, thereby; and
a step of covering a surface of said hydrophilic liquid in said housing chamber with a hydrophobic liquid, by spreading a hydrophobic liquid arranged on the rear side of the moving direction of said wiping member onto said main surface by moving said wiping member.

2. The method for distributing the magnetic bead according to claim 1, wherein
said main surface is hydrophobized, and
said inner surface of said housing chamber is hydrophilized.

3. The method for distributing the magnetic bead according to claim 1, wherein
said wiping member is cylindrical with a central axis along said main surface.

4. The method for distributing the magnetic bead according to claim 3, wherein
said wiping member comprises a cylindrical main body and a covering layer formed on said outer circumferential surface of said main body and having a hardness lower than that of said outer circumferential surface.

5. The method for distributing the magnetic bead according to claim 1, further comprises
a step of forming an annular groove surrounding said housing chamber and placing a liquid stacking frame surrounding said housing chamber on said main surface;
a step of forming a hydrophobic liquid layer made of a hydrophobic liquid on said main surface within said liquid stacking frame; and
a step of introducing a hydrophilic liquid into said annular groove and causing said hydrophilic liquid to overflow said annular groove to form a hydrophilic liquid layer on said hydrophobic liquid layer.

6. A magnetic bead distribution device, comprising
a distribution magnetic bead into a plurality of housing chamber of a substrate having a main surface on which said housing chamber are formed, wherein
a wiping member having a shape that conforms to said main surface, and
a magnet placed on a side of said substrate that is opposite from said wiping member,
wherein said wiping member is movable forward along said main surface carrying a hydrophilic liquid containing a plurality of magnetic bead, and
said magnet is simultaneously movable forward along said wipe member while exerting a magnetic force on said magnetic bead.
